(19) United States
(12) Patent Application Publication
(10) Pub. No.: US 2014/0148594 A1
(43) Pub. Date: May 29, 2014

(54) Azaporphyrins And Applications Thereof

(71) Applicants: Jian Li, Phoenix, AZ (US); Zixing Wang, Shanghai (CN)

(72) Inventors: Jian Li, Phoenix, AZ (US); Zixing Wang, Shanghai (CN)

(21) Appl. No.: 14/152,580

(22) Filed: Jan. 10, 2014

Related U.S. Application Data
(63) Continuation of application No. 13/255,556, filed on Dec. 19, 2011, now abandoned.
(60) Provisional application No. 61/159,556, filed on Mar. 12, 2009.

(51) Int. Cl. H01L 51/00

(52) U.S. Cl. CPC H01L 51/0078
USPC 540/121

(57) ABSTRACT

Azaporphyrins that are useful in a variety of optical and electro-optical devices, including photo-absorbing and emitting devices, are disclosed.

### BACKGROUND

1. Technical Field

The present disclosure relates to azaporphyrins, and specifically to π-conjugated azaporphyrins and methods of making and using such azaporphyrins, including optical and electro-optical devices comprising same.

2. Technical Background

Organic compounds with delocalized conjugated π-electrons exhibit unique optical properties and are relatively easy to process into devices. Such compounds are thus ideally suited for use in a wide variety of optical and electro-optical devices, including photo-absorbing devices such as solar- and photo-sensitive devices, photo-emitting devices, such as organic light emitting diodes (OLEDs), or devices capable of both photo-absorption and emission. Much research has been devoted to the discovery and optimization of organic materials for use in optical and electro-optical devices. Generally, research in this area aims to accomplish a number of goals, including improvements in absorption and emission efficiency, as well as improvements in processing ability, among others.

Despite significant advances in research devoted to optical and electro-optical materials, many current devices comprising organic materials have yet to be optimized. Many organic materials currently used in optical and electro-optical devices have a number disadvantages, including poor processing ability, inefficient emission or absorption, and less than ideal stability, among others. Thus, a need exists for new organic materials that can exhibit improved performance in optical and electro-optical devices. This need and other needs are satisfied by the compositions and methods of the present invention.

### SUMMARY

In accordance with the purpose(s) of the invention, as embodied and broadly described herein, this disclosure, in one aspect, relates to azaporphyrins that can be useful in a variety of optical and electro-optical devices.

In one aspect, the azaporphyrins disclosed herein are represented by the following formula:

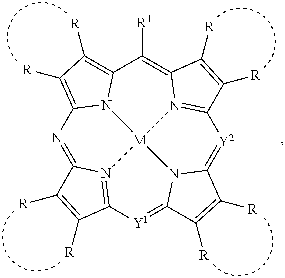

wherein M is a transition metal, such as, for example, Zn²⁺, Cu²⁺, Pd²⁺, Pt²⁺, Fe²⁺, Co²⁺, Ni²⁺, or Mg²⁺; wherein Y¹ and Y² are independently N or CR⁶, wherein R⁶ is optionally substituted aryl; wherein R¹ is hydrogen or optionally substituted aryl; and wherein each general “R” group independently comprises an organic or inorganic residue, such as alkyl, alkenyl, alkynyl, aryl, cyano, halogen, among others; wherein - - - - - is an optional bond, such that two adjacent “R” groups can form a ring (such as an aryl) together with the carbon to which they are attached.

In a further aspect, the azaporphyrin is represented by the formula:

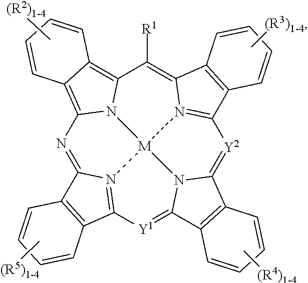

wherein M is Zn²⁺, Cu²⁺, Pd²⁺, Pt²⁺, Fe²⁺, Co²⁺, Ni²⁺, or Mg²⁺; wherein Y¹ and Y² are independently N or CR⁶, wherein R⁶ is optionally substituted aryl; wherein R¹ is hydrogen or optionally substituted aryl; and wherein each of R², R³, R⁴, and R⁵ independently comprises four substituents independently selected from hydrogen, halide, and aryl, or wherein two adjacent substituents form an optionally substituted aryl ring together with the carbon to which they are attached, with the two other substituents being selected from hydrogen, halide, and aryl.

Also disclosed are optical and electro-optical devices comprising the azaporphyrins of the present invention. In various aspects, light emitting devices, photovoltaic devices, and solar devices, among others, comprise one or more disclosed azaporphyrins.

### BRIEF DESCRIPTION OF THE FIGURES

The accompanying figures, which are incorporated in and constitute a part of this specification, illustrate several aspects and together with the description serve to explain the principles of the invention.

FIGS. 1A-E show schematics of device applications of the disclosed metal azaporphyrins: A) donor-type materials for photovoltaic cells; B) absorbers for dye-sensitized solar cells; C) emitters for red and near infra-red organic light emitting diodes; D) absorbers and re-emitters for organic concentrators (i.e. a large area of organic film as collector of sunlight for small-size and high efficiency inorganic PVs); E) absorbers for a hydrogen generation system; all in accordance with the various aspects of the present invention.

FIG. 2A shows an absorption spectrum of III-Zn-2 in dichloromethane, in accordance with the various aspects of the present invention,

FIG. 2B shows a photoluminescence spectrum of III-Zn-2 in dichloromethane, in accordance with the various aspects of the present invention.

FIG. 3A shows an absorption spectrum of I-Zn-2 in dichloromethane, in accordance with the various aspects of the present invention.

FIG. 3B shows a photoluminescence spectrum of I-Zn-2 in dichloromethane, in accordance with the various aspects of the present invention.

FIG. 4A shows an absorption spectrum of IV-Zn-2 in dichloromethane, in accordance with the various aspects of the present invention.

FIG. 4B shows a photoluminescence spectrum of IV-Zn-2 in dichloromethane, in accordance with the various aspects of the present invention.

FIG. 5A shows an absorption spectrum of III-H2-2 in dichloromethane, in accordance with the various aspects of the present invention.

FIG. 5B shows a photoluminescence spectrum of III-H2-2 in dichloromethane, in accordance with the various aspects of the present invention.

FIG. 6A shows an absorption spectrum of I-H2-2 in dichloromethane, in accordance with the various aspects of the present invention.

FIG. 6B shows a photoluminescence spectrum of I-H2-2 in dichloromethane, in accordance with the various aspects of the present invention.

FIG. 7 illustrates the current voltage characteristics of a device comprising ITO/ZnTABP (IV-Zn-2)×nm/C60(30 nm)/PTCDI(10 nm)/BCP(14 nm)/Al under 1 sun condition (AM1.5 at 100 mW/cm²).

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

### DEFINITIONS

As used in the specification and the appended claims, the singular forms “a,” “an” and “the” include plural referents unless the context clearly dictates otherwise. Thus, for example, reference to “an azaporphyrin” includes mixtures of two or more azaporphyrins.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not.

As used herein, the term “azaporphyrin” refers to a porphyrin analog wherein at least one meso position is aza substituted, i.e., wherein a methine bridge (—CH—) (meso position) of a porphyrin is substituted with a nitrogen atom (aza substitution). The azaporphyrin derivatives can be monoazaporphyrins, diazaporphyrins, triazaporphyrins, tetraazaporphyrins, or derivatives thereof, such as tetrabenzo-substituted derivatives disclosed herein. The prefixes mono, di, tri, and tetra, as used herein to modify “azaporphyrin,” refer to the number of aza substitutions at the meso position(s). Also included within the term “azaporphyrins” are derivatives of azaporphyrins, which may not strictly correspond to an azaporphyrin structure, but are structurally related.

A chemical term used herein to describe a compound or chemical residue refers to the compound or residue regardless of whether the compound or residue is actually obtained from the chemical species in the chemical term used to describe the compound or residue. Thus, for example, the term “azaporphyrin” does not imply that the compound in reference was made from porphyrin. Likewise, a disclosed “derivative” or “analog” only implies a relation, for example a structural relation, between the derivative or analog and the chemical species or term used to modify the term “derivative” or “analog.” Thus, for example, an “azaporphyrin derivative” does not imply that the azaporphyrin derivative was derived from an azaporphyrin. However, it is understood that a disclosed azaporphyrin derivative can, in some aspects, be derived from an azaporphyrin.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

An “optionally substituted” compound refers to a compound that can be, but does not have to be, substituted with a substituent, such as those described below or other substituents that are not specifically disclosed but would not interfere with the desired function of the compound.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

“Alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

One or more of the above described chemical terms may not be specifically referenced in the context of a disclosed compound, but it is contemplated that one or more of the above defined groups can be present as a substituent on a disclosed compound, unless the context clearly dictates otherwise.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

In one aspect, the present invention relates to azaporphyrins which are useful as components of an optical or electro-optical device. The disclosed azaporphyrins exhibit a variety of useful properties, including for example, efficient absorption, emission, or absorption and emission, making them ideally suited for use in an optical or electro-optical device. In one aspect, the disclosed compounds are hybrids of porphyrin-based compounds and phthalocyanines (tetrabenzotetraazaporphyrin), and as such exhibit a variety of advantages unique to both porphyrins and phthalocyanines, including even synergistic advantages unrealized with a porphyrin or phthalocyanine alone. For example, the disclosed azaporphyrins show strong absorption in the visible region of the electromagnetic spectrum, as exhibited by many phthalocyanines. Additionally, the disclosed azaporphyrins are also highly emissive, much like many porphyrin-based compounds. It follows that the disclosed azaporphyrins are ideally suited for use in a wide range of applications, including without limitation light emitting diodes, organic thin solar cells, dye-sensitized solar cells, organic concentrators, and solar hydrogen generation systems, as discussed herein.

The azaporphyrins disclosed herein can be monoazaporphyrins, diazaporphyrins, triazaporphyrins, tetraazaporphyrins, or derivatives thereof, including for example, benzoazaporphyrins. Generally, the compounds are represented by the following formula:

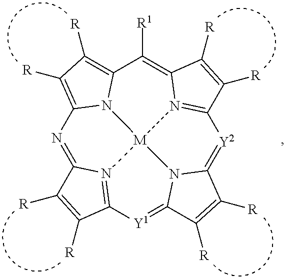

wherein M is a transition metal, such as Zn²⁺, Cu²⁺, Pd²⁺, Pt²⁺, Fe²⁺, Co²⁺, Ni²⁺, or Mg²⁺; wherein Y¹ and Y² are independently N or CR⁶, wherein R⁶ is optionally substituted aryl; wherein R¹ is hydrogen or optionally substituted aryl; and wherein each general “R” group independently comprises an organic or inorganic residue, such as alkyl, alkenyl, alkynyl, aryl, cyano, halogen, among others; wherein - - - - - is an optional bond, such that two adjacent “R” groups can form a ring (such as an aryl) together with the carbon to which they are attached.

In a further aspect, the azaporphyrin is a tetrabenzoazaporphyrin. Examples of tetrabenzoazaporphrins include without limitation tetrabenzomonoazaporphyrin, tetrabenzodiazaporphyrin, tetrabenzotriazaporphyrin, tetrabenzotetraazaporphyrin (also known as Phthalocyanine), and derivatives thereof. Exemplary tetrabenzoazaporphyrins are represented by the following formula:

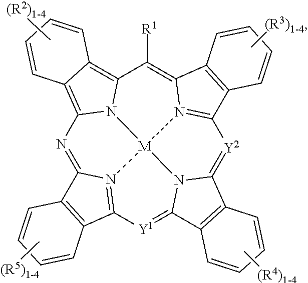

wherein M is Zn²⁺, Cu²⁺, Pd²⁺, Pt²⁺, Fe²⁺, Co²⁺, Ni²⁺, or Mg²⁺; wherein Y¹ and Y² are independently N or CR⁶, wherein R⁶ is optionally substituted aryl; wherein R¹ is hydrogen or optionally substituted aryl; and wherein each of R², R³, R⁴, and R⁵ independently comprises four substituents independently selected from hydrogen, halide, and aryl, or wherein two adjacent substituents form an optionally substituted aryl ring together with the carbon to which they are attached, with the two other substituents being selected from hydrogen, halide, and aryl.

In a specific aspect, the azaporphryin comprises one or more of Zn²⁺, Cu²⁺, Pd²⁺, or Pt²⁺. For example, in the formulae above or below, M can be Zn²⁺, Cu²⁺, Pd²⁺, or Pt²⁺. In a further aspect of a disclosed azaporphyrin formula, one or more of Y¹ or Y² is N. Thus, as described above, if one of Y¹ or Y² is N, the compound is a diazaporphyrin. Likewise, if both of Y¹ and Y² are N, the compound is a triazaporphyrin, and so on. In another specific aspect, R¹ is hydrogen. In a different aspect, R¹ is phenyl, for example bromophenyl.

In other specific aspects, one or more of R², R³, R⁴ or R⁵ independently comprises two adjacent substituents forming an optionally substituted aryl ring together with the carbon to which they are attached, with the two other substituents on the benzo-ring being selected from hydrogen, halide, and aryl. In a different aspect, one or more of R², R³, R⁴, and R⁵ independently comprises four hydrogens.

In a further aspect, when CR⁶ is present as Y¹ or Y² or both, R⁶ can comprise a variety of substituents, such as aryl. In one aspect, R⁶ is phenyl, such as, for example, bromophenyl.

In one aspect, the azaporphyrin is represented by one or more of the following formulae:

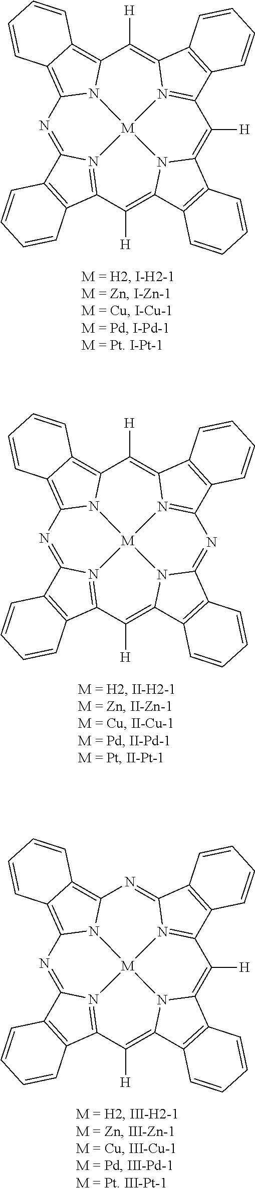

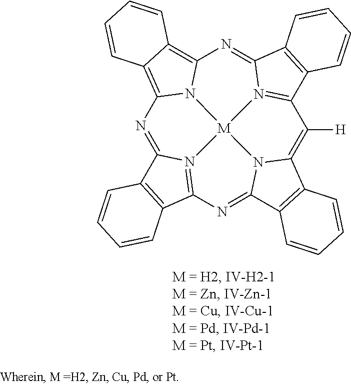

In another aspect, the azaporphyrin is represented by one or more of the following formulae:

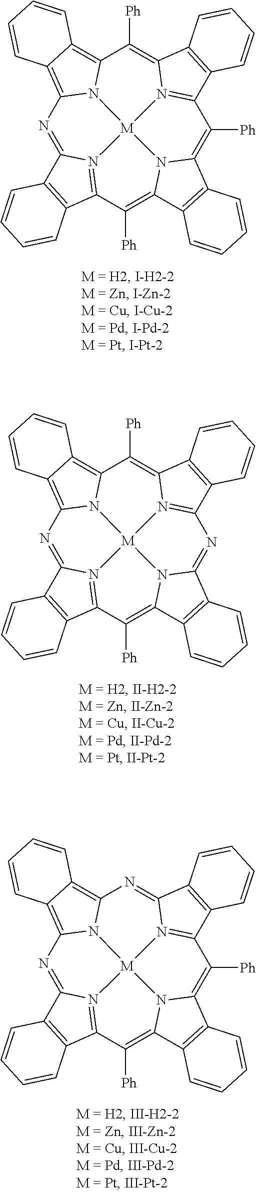

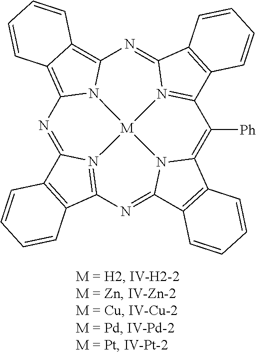

In another specific aspect, the azaporphyrin is represented by:

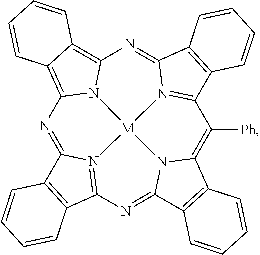

wherein M comprises zinc.

In another aspect, the azaporphyrin is represented by one or more of the following formulae:

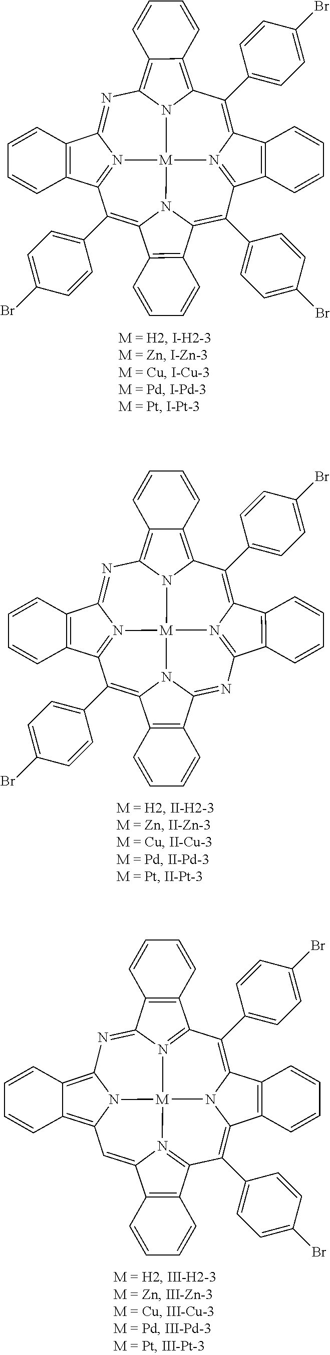

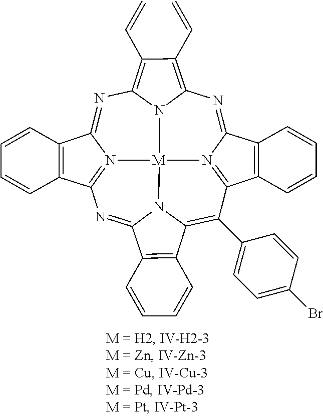

In another aspect, the azaporphyrin is represented by one or more of the following formulae:

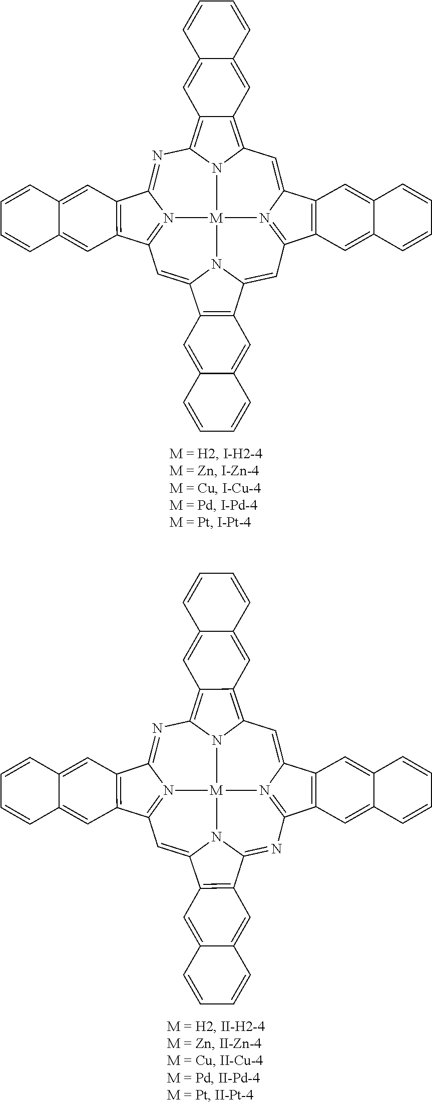

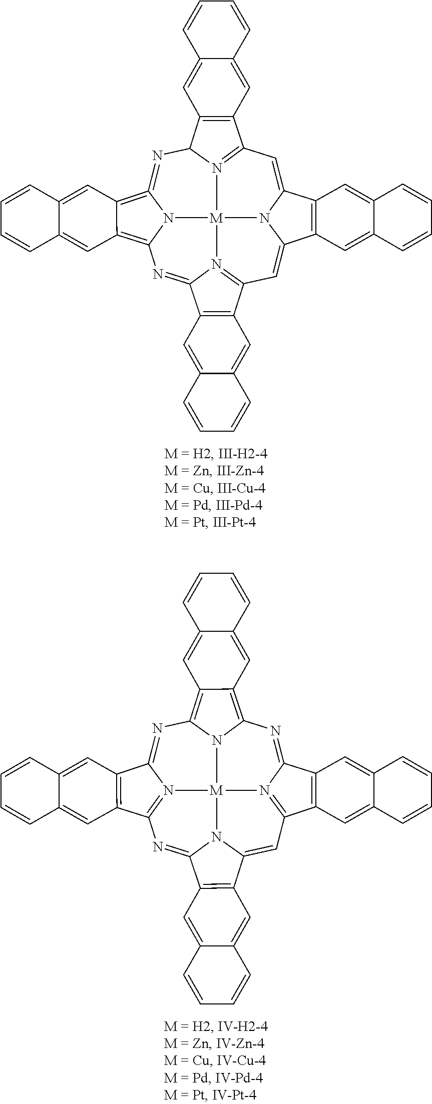

In another aspect, the azaporphyrin is represented by one or more of the following formulae.

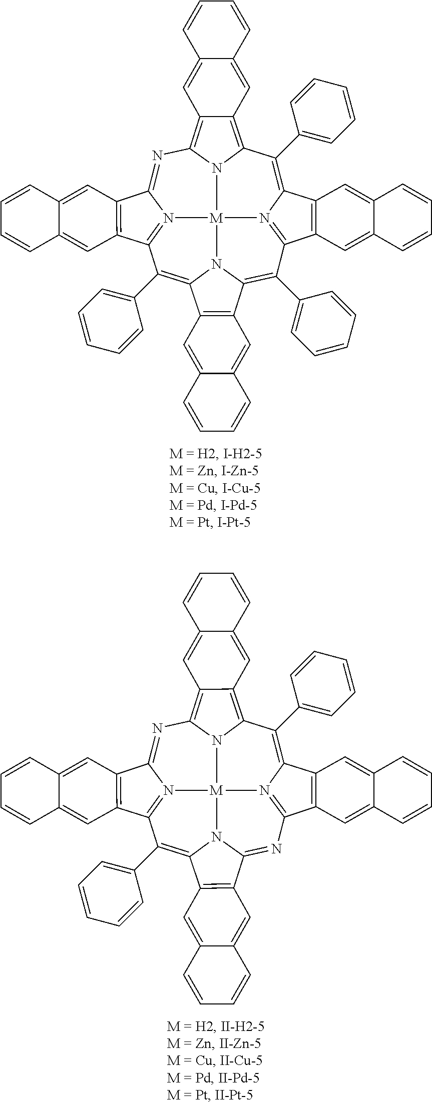

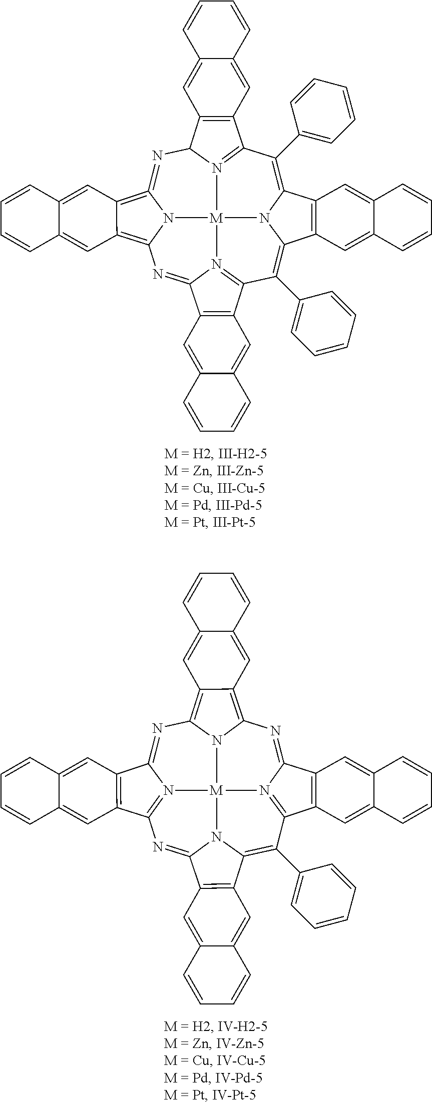

In another aspect, the azaporphyrin is represented by one or more of the following formulae:

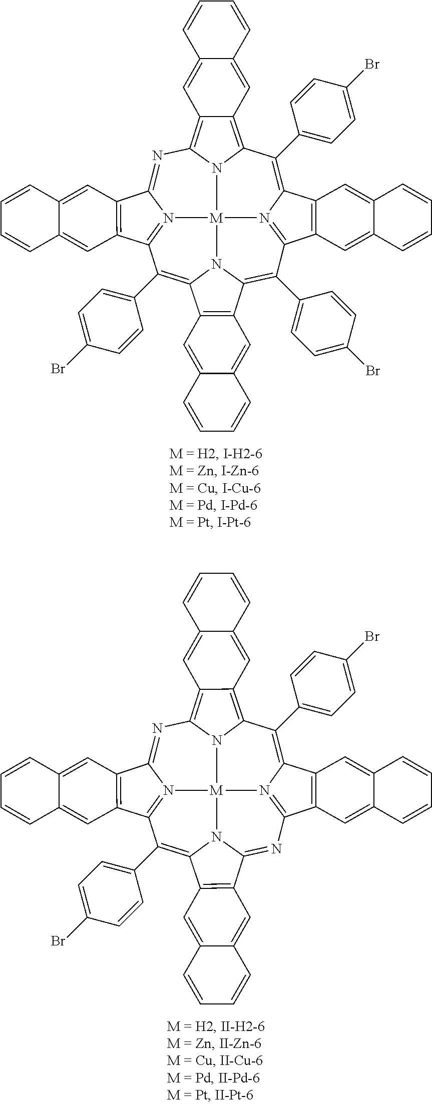

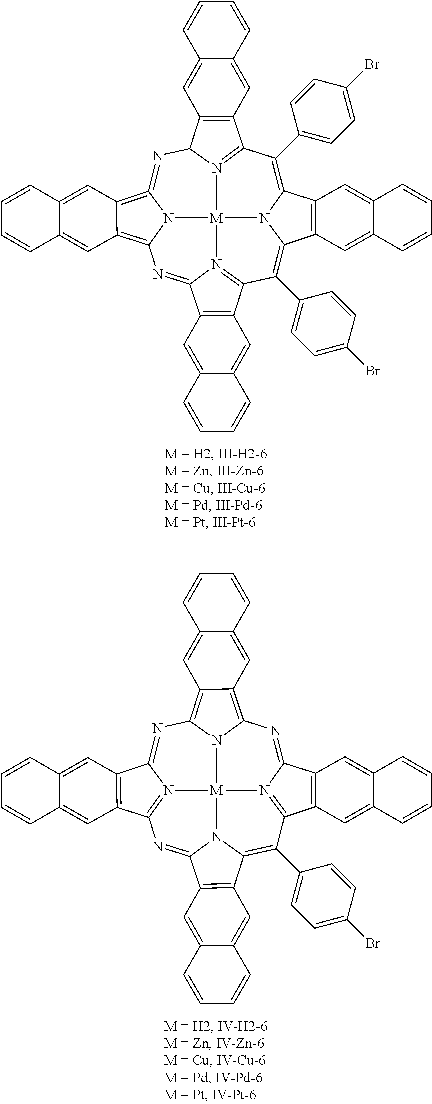

In another aspect, the azaporphyrin is represented by one or more of the following formulae:

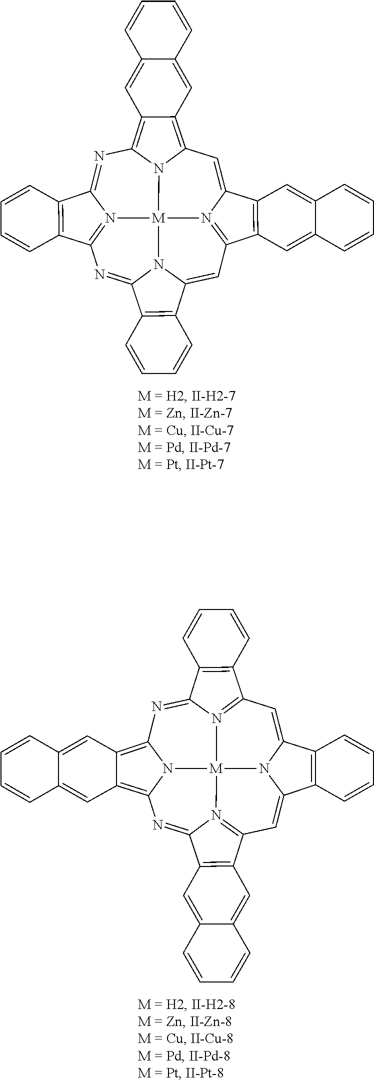

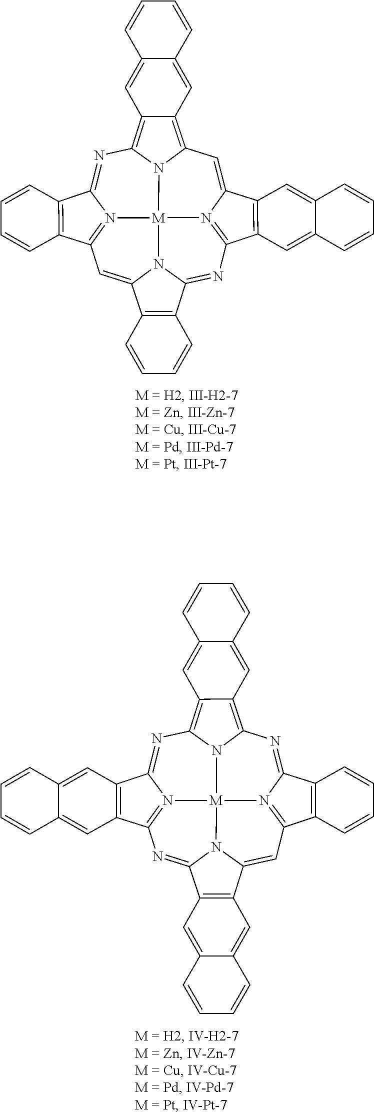

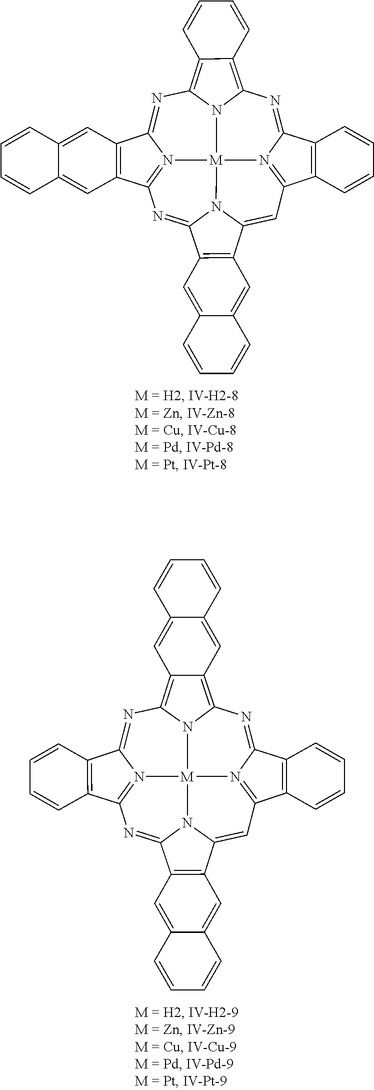

In another aspect, the azaporphyrin is represented by one or more of the foilowing formulae.

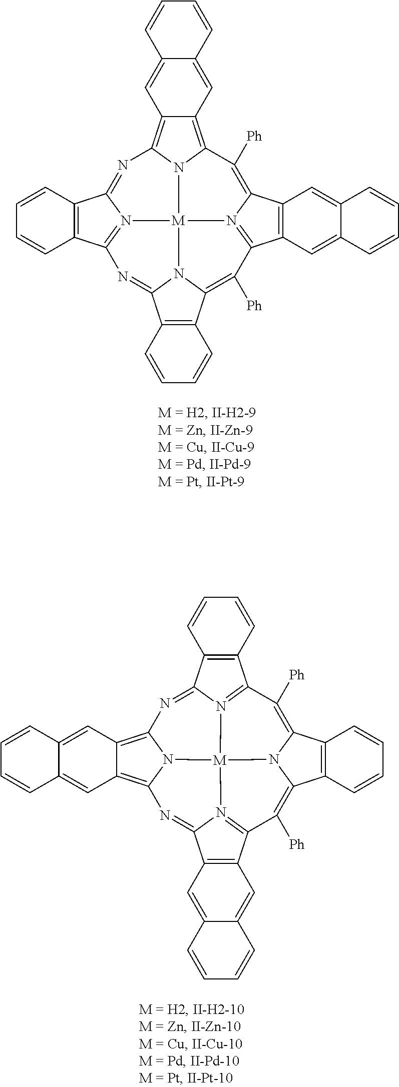

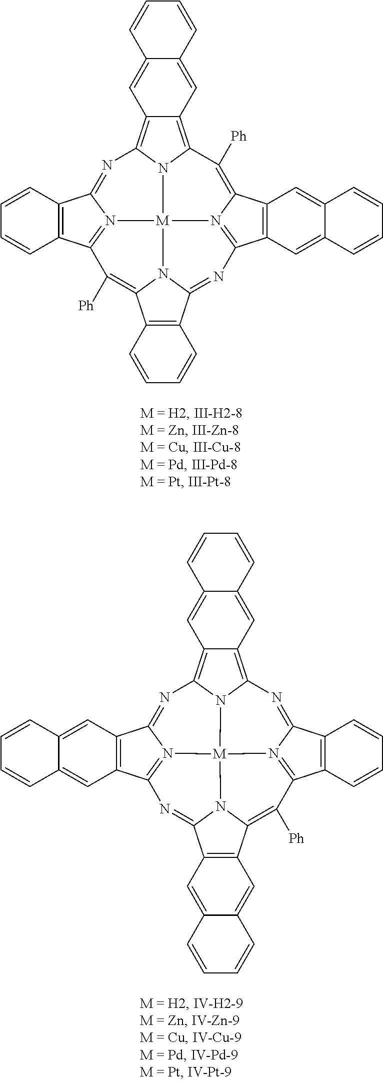

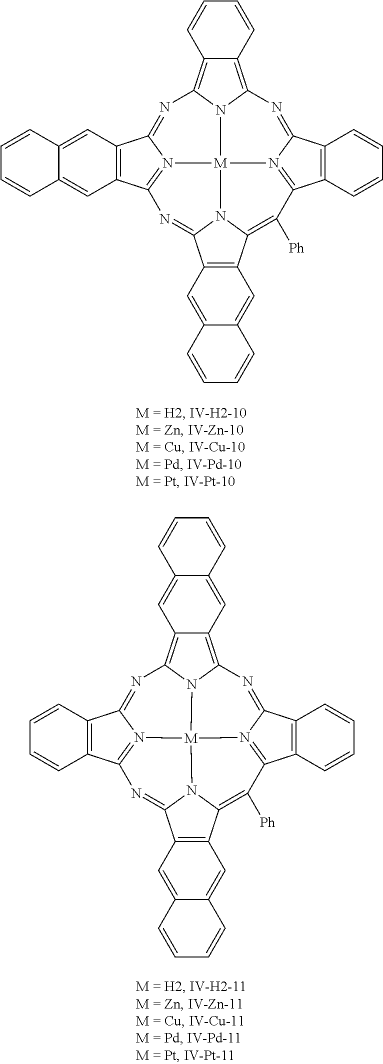

In another aspect, the azaporphyrin is represented by one or more of the following formulae:

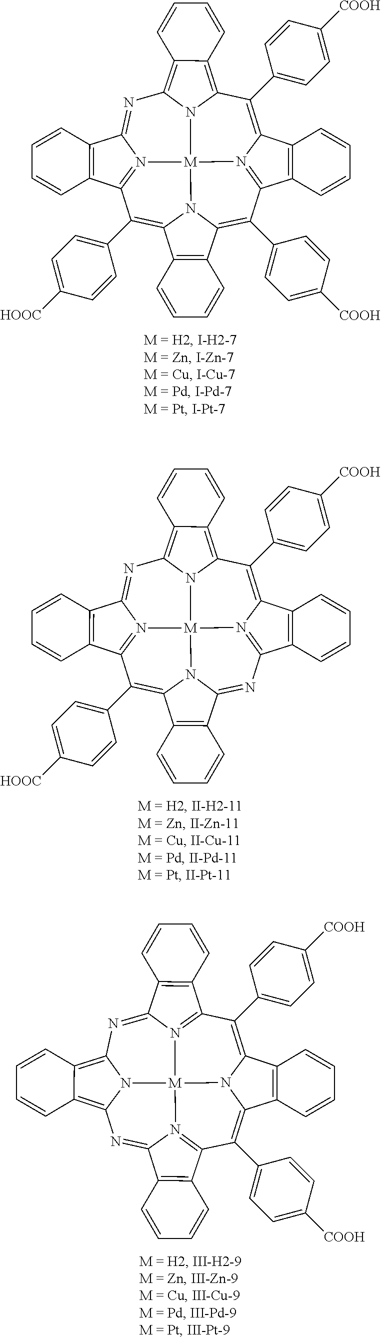

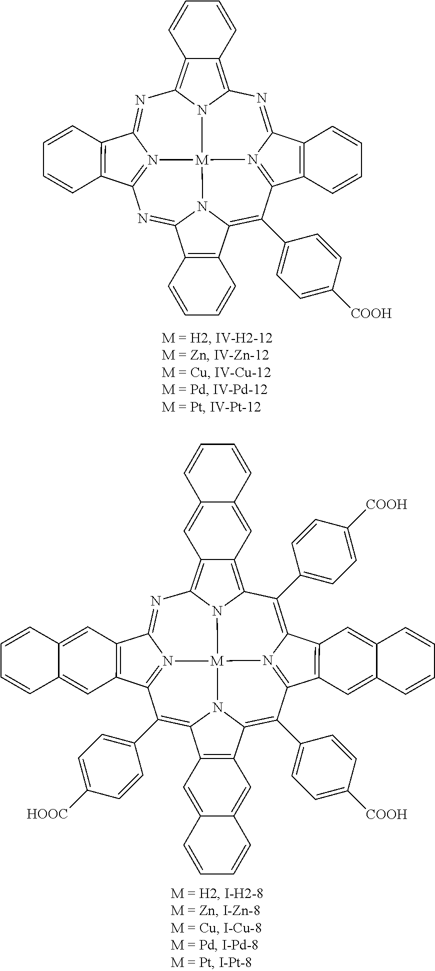

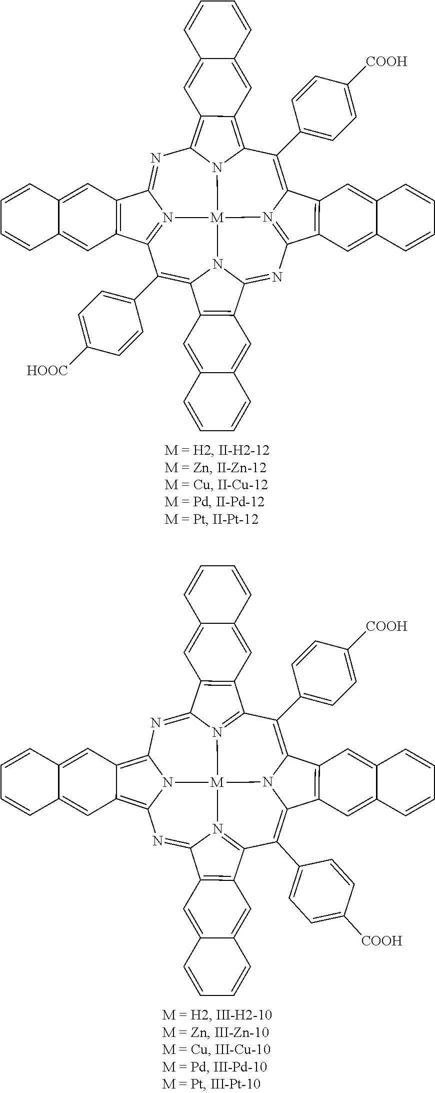

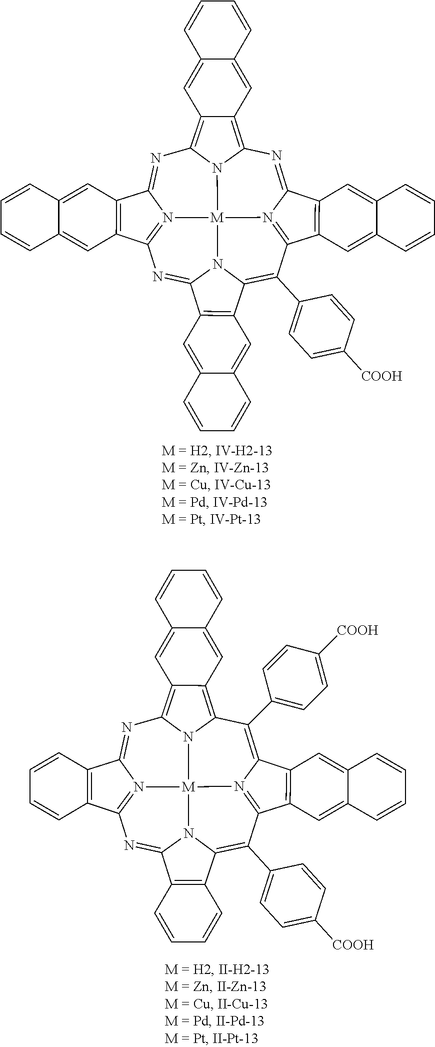

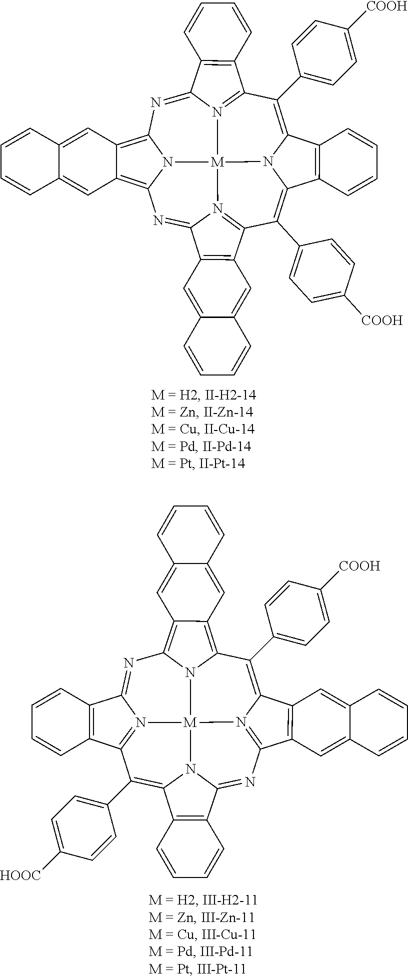

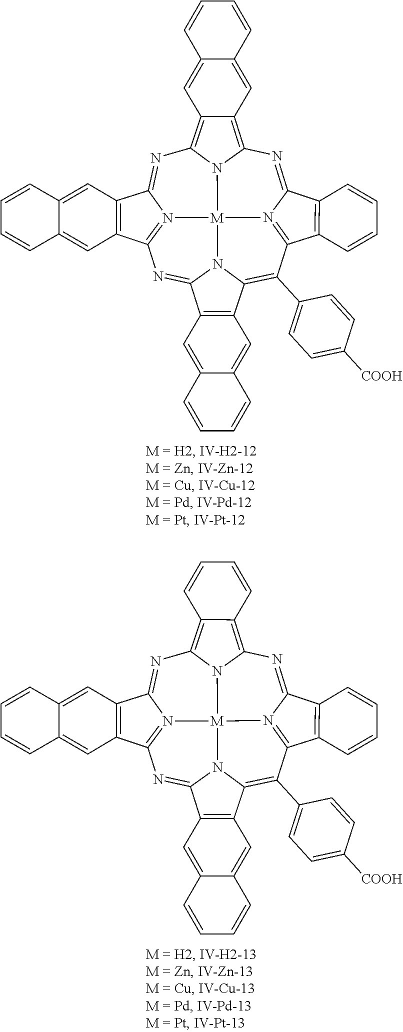

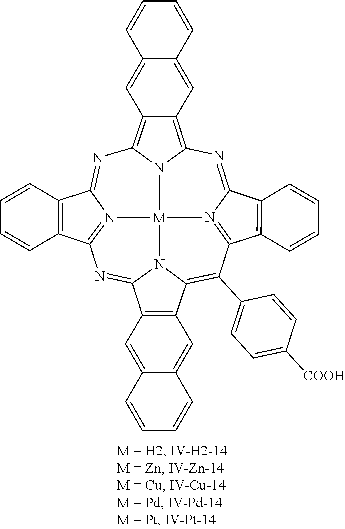

In one aspect, the azaporphyrin is not represented by:

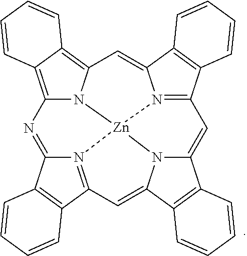

In one aspect, the azaporphyrin is not represented by:

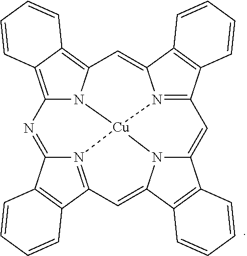

In one aspect, the azaporphyrin is not represented by:

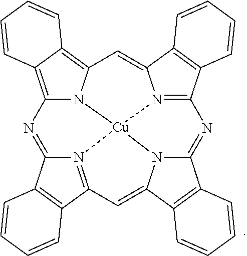

In one aspect, the azaporphyrin is not represented by:

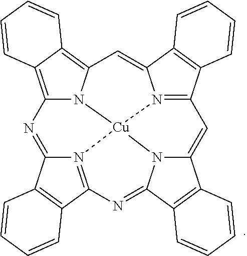

In one aspect, the azaporphyrin is not represented by:

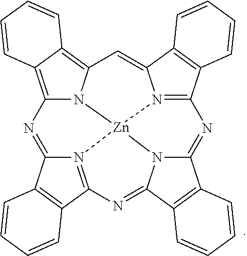

In one aspect, the azaporphyrin is not represented by:

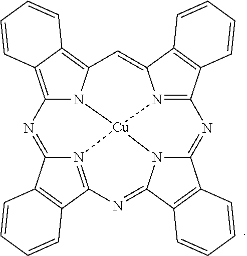

In one aspect, the azaporphyrin is not represented by:

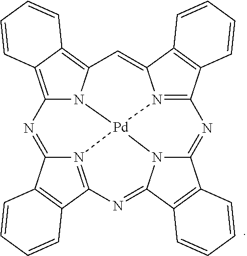

In one aspect, the azaporphyrin is not represented by:

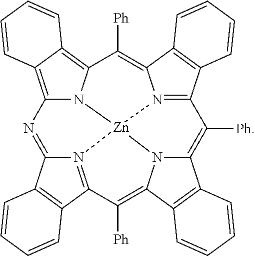

In one aspect, the azaporphyrin is not represented by:

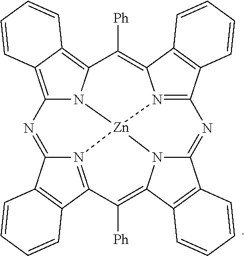

In one aspect, the azaporphyrin is not represented by:

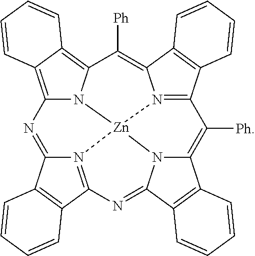

In one aspect, the azaporphyrin is not represented by:

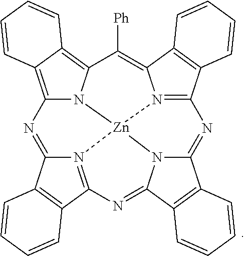

In a further aspect of a formula above, each of R², R³, R⁴, and R⁵ independently comprises four hydrogens, provided that M is not Zn, Cu, or Pd.

The disclosed azaporphyrins can be made using a variety of synthetic protocols, either known or disclosed herein. Exemplary synthetic methods generally start with a reaction of one or more of the four corner π-congugated groups of the azaporphyrin. Suitable starting materials for forming the azaporphyrin ring or precursor therefore include without limitation phthalic acid, phthalonitrile, o-cyanobenzamide, phthalanhydride, phthalimide, diiminoisoindole, including derivatives and combinations thereof. From these starting materials or other starting materials, the azaporphyrin ring can be provided using a one-pot method (i.e. wherein the ring is fully formed) or a step-wise method (i.e. wherein each corner of the azaporphyrin is in a sequence).

In some aspects, the azaporphyrin is provided by reacting an amine or imine containing compound, such as an indole or other starting material discussed above, with a corresponding acid, activated ester, or other suitable electrophile to provide the imine (or aza functionality). This protocol can be used to provide the azaporphyrin in a one-pot reaction. In another aspect, a dimer formed during such a reaction can be further reacted with itself to form the fully cyclized azaporphryin. In some aspects, it can be preferable to perform the cyclization reaction under dilute conditions (thermodynamic conditions) so as not to induce unwanted kinetic reactions producing trimers, oligimers, and the like. A metal can be inserted during or after the formation of the azaporphyrin ring using metal insertion reactions, such as oxidation or reduction reactions. In some aspects, an inserted metal can be exchanged with another metal to provide a different metal center using a further metal insertion or exchange reaction.

In another aspect, an azaporphyrin or the products of an azaporphyrin synthesis procedure can optionally be subjected to one or more purification steps, such as, for example, a chromatographic separation. In such an aspect, one or more desired azaporphyrin products can be separated and/or optionally isolated from any other reaction products or byproducts that may be present.

The disclosed azaporphyrins, as discussed above, can be used in a variety of applications, including without limitation optical and electro-optical devices. Exemplary devices include without limitation light emitting diodes, organic thin solar cells, dye-sensitized solar cells, organic concentrators, and solar hydrogen generation systems. In one aspect, a disclosed azaporphyrin can be used a portion of an organic thin solar cell. In one aspect, a disclosed azaporphyrin can be used a portion of a dye-sensitized solar cell. In one aspect, a disclosed azaporphyrin can be used a portion of an organic concentrator. In one aspect, a disclosed azaporphyrin can be used a portion of a solar hydrogen generation system. In another aspect, a disclosed azaporphyrin can form a light emitting layer or a portion thereof. In another aspect, a disclosed azaporphyrin can form a light absorbing layer or a portion thereof. It should be appreciated that other components can optionally be mixed with or used in a same layer as an azaporphyrin. It should also be appreciated that other layers can optionally be provided in a device, and one of skill in the art could readily select an appropriate composition for such a device. The disclosed azaporphyrins can be processed into such a device using methods known in the art. In one aspect, any processing method for incorporating an azaporphyrin of the present invention into a device does not damage or adversely affect the light absorbing or light emitting properties of the azaporphyrin. Depending on the desired application, the disclosed azaporphyrins can be attached to a surface, e.g. covalently attached, or polymerized together to afford a polymer which can in some aspects provide for a better ability to process the azaporphyrin into a device.

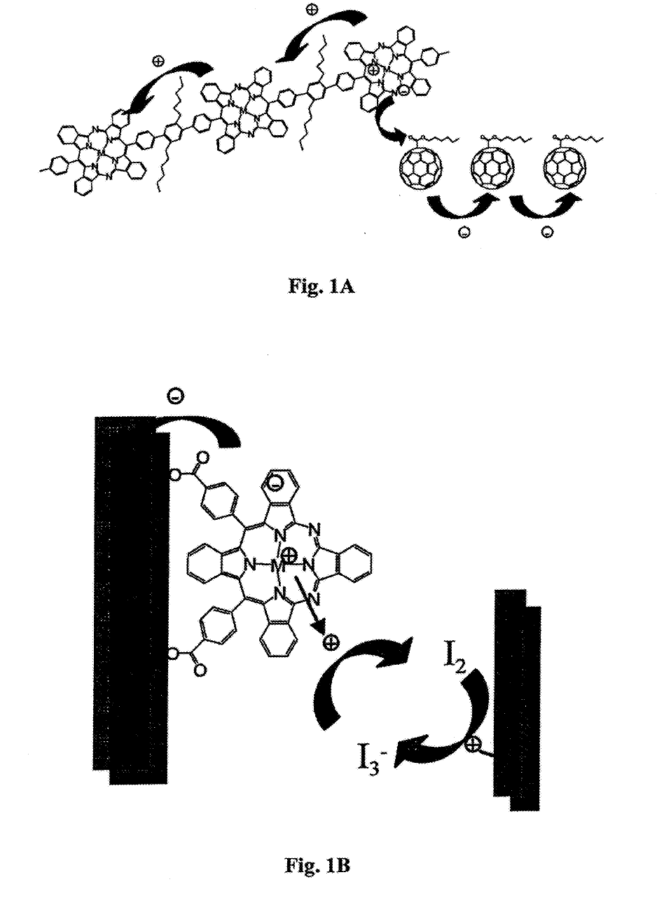

With reference to FIGS. 1A-1E, a variety of general device schematics are shown, comprising exemplary azaporphyrins disclosed herein. With reference to FIG. 1A, a polymer of a disclosed azaporphyrin can be used as a charge donor-type material for a photovoltaic cell. With reference to FIG. 1B, a disclosed azaporphyrin can be covalently attached to a surface and used as a photon-absorbing material for a solar cell, such as a dye-sensitized solar cell. With reference to FIG. 1C, a disclosed azaporphyrin can be used as the emitting material for an organic light emitting diode, such as a red or near infra-red organic light emitting diode. With reference to FIG. 1D, a disclosed azaporphyrin can be used as both an absorber and re-emitter for an organic light concentrator (e.g., as a large area of a collector of sunlight for small-size and high efficiency inorganic photovoltaics). With reference to FIG. 1E, a disclosed azaporphyrin can be used as an absorber for a hydrogen generation system or other catalytic system.

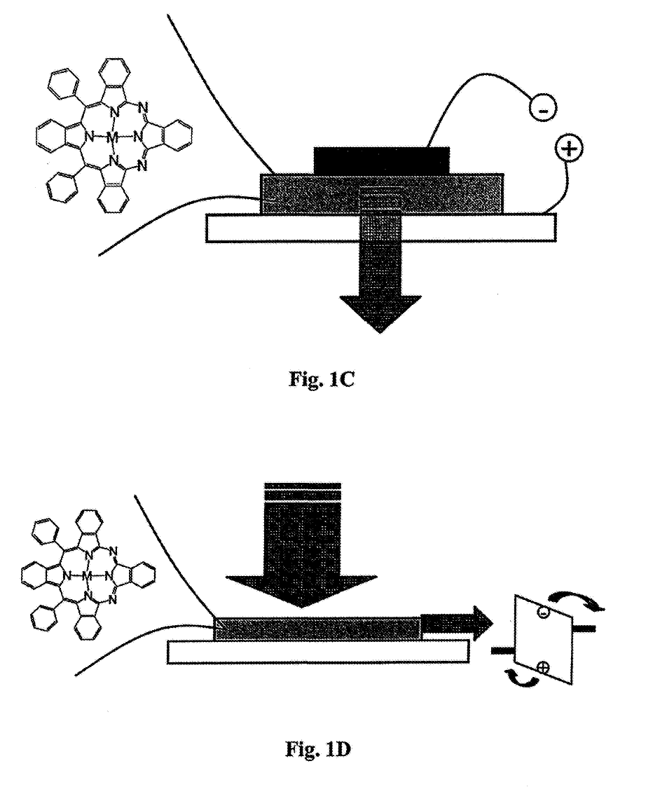

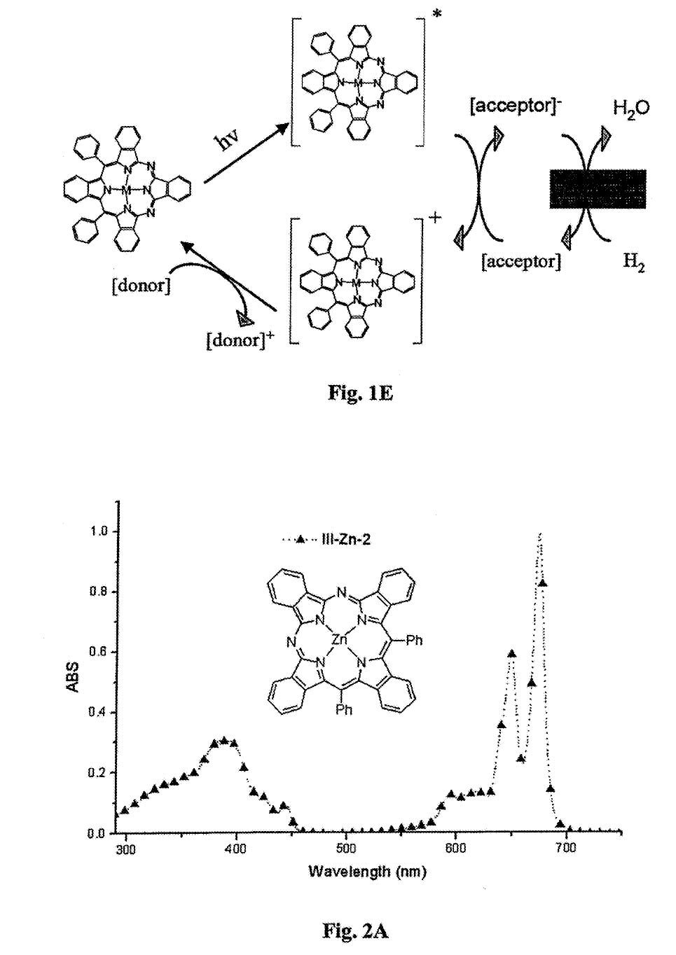

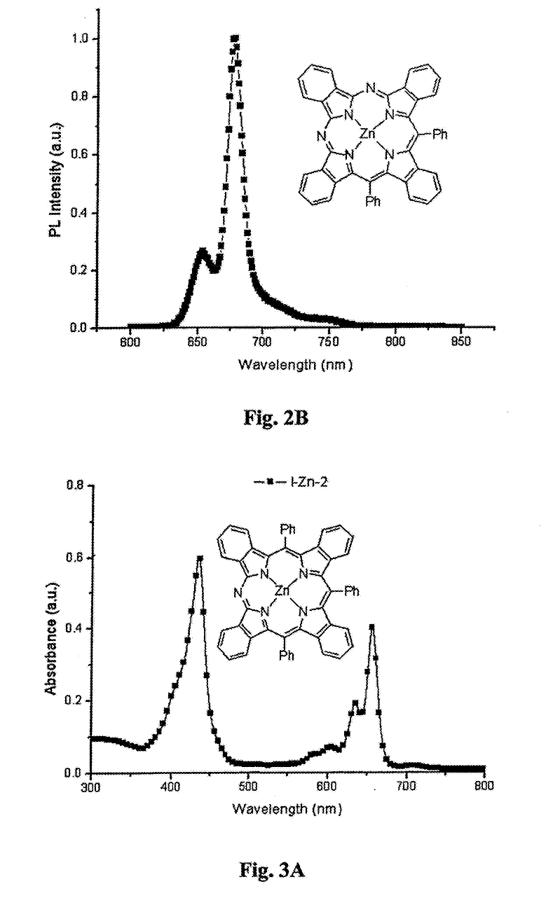

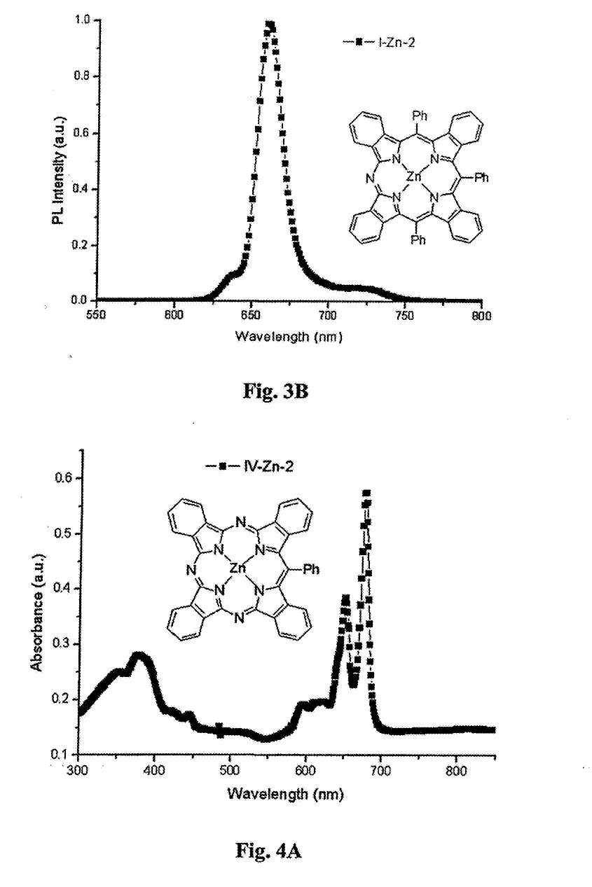

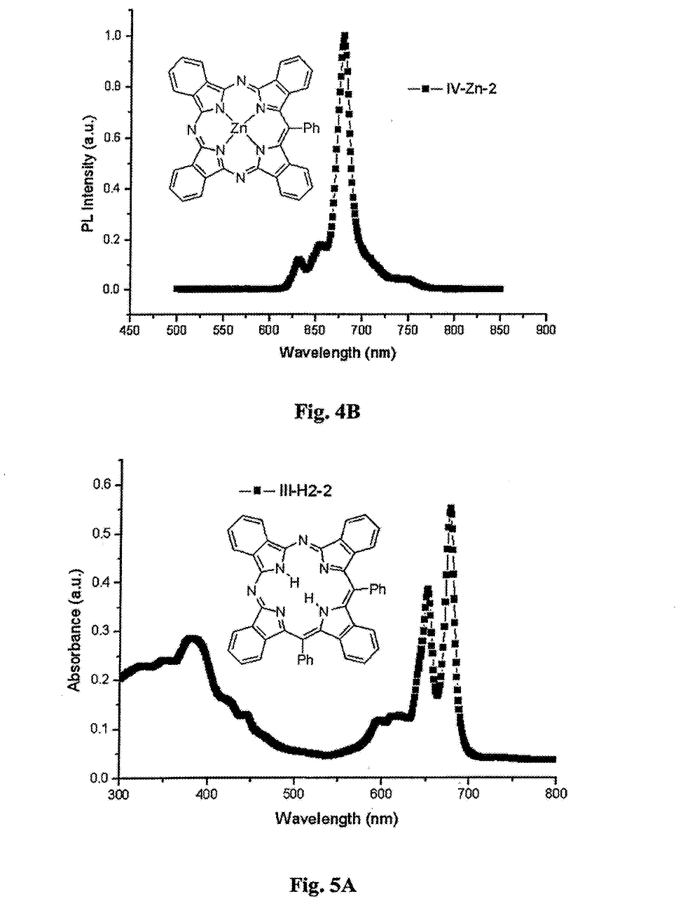

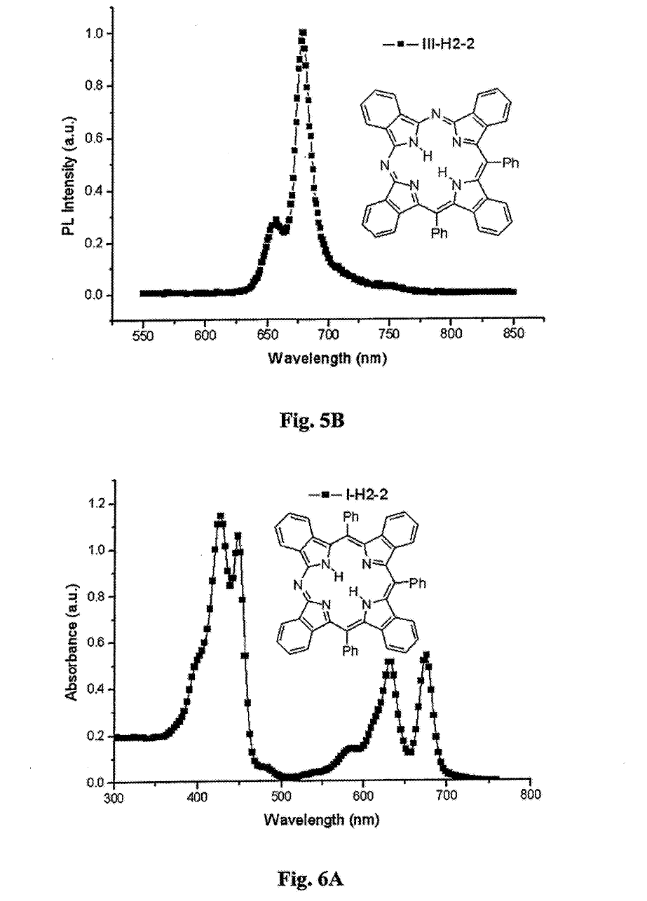

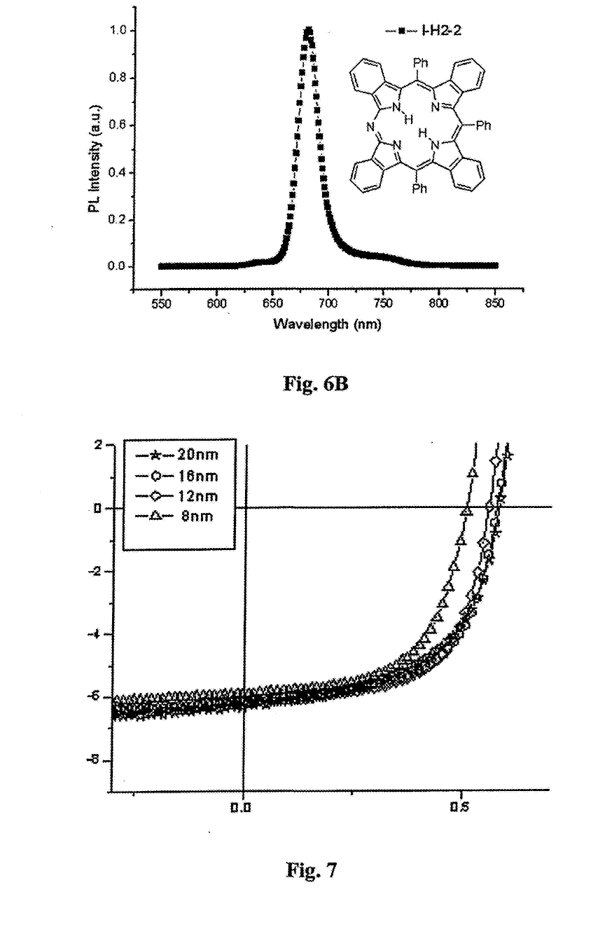

As can be seen from FIGS. 2A-4B, exemplary azaporphyrins disclosed herein exhibit useful optical properties, including without limitation, strong absorption in a variety of spectral regions, including blue, red, and infra-red regions. It will be apparent that the absorption properties of the disclosed azaporphyrins is related to the electronic structure of the azaporphyrin, which can be modulated through the introduction of various functional groups (such as electron withdrawing or donating groups) at a variety of locations of the azaporphyrin ring, in addition to changing the metal center. As an example, the azaporphyrin shown in FIGS. 2A and 2B, which is a diphenyl substituted diazatetrabenzoporphyrin, exhibits strong absorption and emission near the red region of the spectrum, while the azaporphyrin shown in FIGS. 3A and 3B, which is a triphenyl monoazatetrabenzoporphyrin, exhibits two strong absorption peaks in the blue and near-red regions and emission in the near-red to red region. The ability to tailor the absorption and emission of the disclosed azaporphyrins allows for the design of different compounds for different optical and electro-optical applications. As a reference and control for the exemplary azaporphyrins shown in FIGS. 2A-4B, FIGS. 5A-6B show absorption and emission spectra of un-metallated azaporphryn rings.

In one aspect, an azaporphyrin can comprise a plurality of a single type of azaporphyrin. In other aspects, an azaporphyrin composition can comprise a mixture of two or more different azaporphyrins. In another aspect, multiple different azaporphyrins can be combined so as to, for example, absorb and/or emit light at a variety of wavelengths.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Synthesis: Exemplary Class of Azaporphyrins

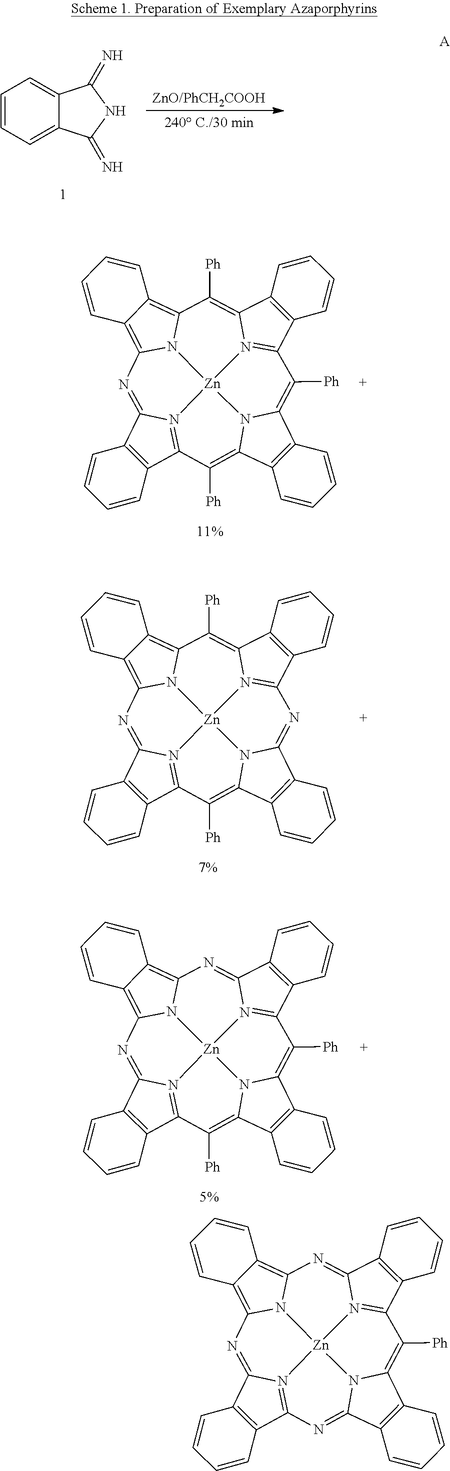

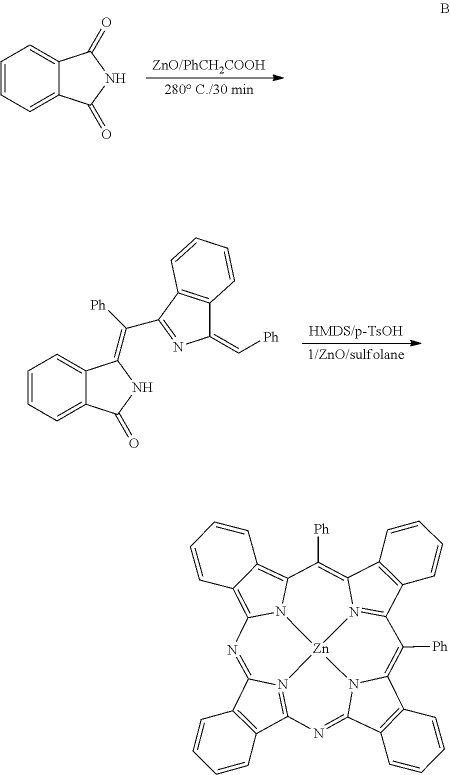

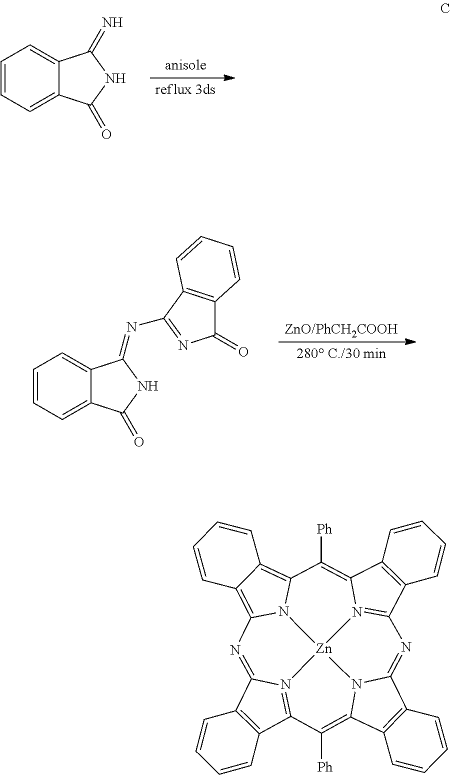

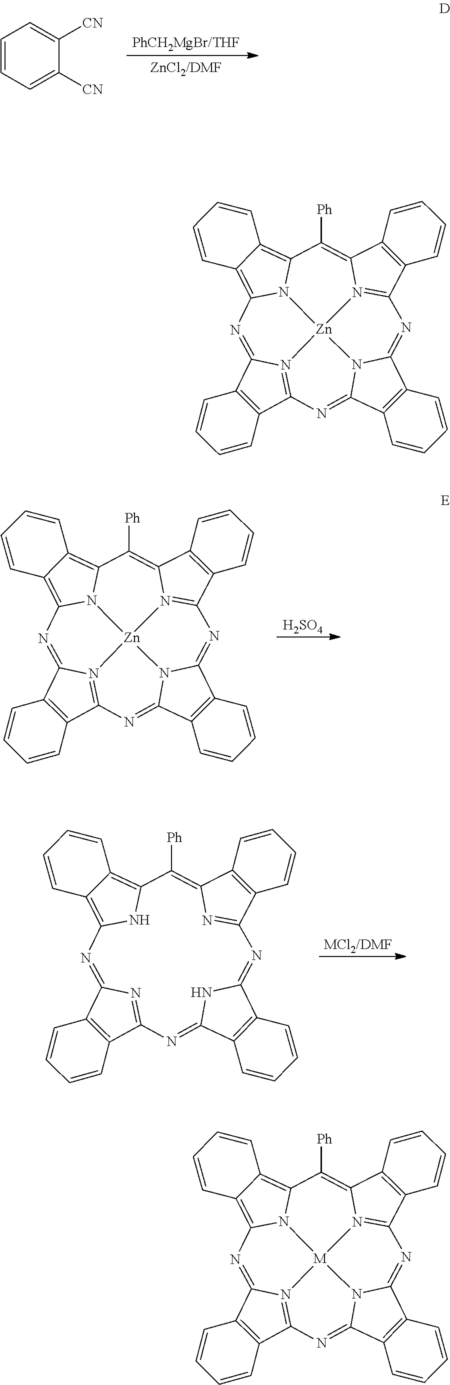

2. Synthesis of I-Zn-2:

A mixture of phthalimide (6 g), phenylacetic acid (5 g), and zinc oxide (3.2 g) was kept at 250° C. for h, after which the melt was cooled, ground, and boiled in 100 mL of 10% HCl for 5 min. The precipitate was filtered off and washed with 200 mL of hot water. The dried solid was purified by chraomatograph. The main red band was collected to produce intermediate. This intermediate was treated with p-toluenesulfuric acid and HMDS (2 eq.) for 10 hours under 110° C. 1,3-diaminoisoindoline (4 eq.), zinc oxide (2 eq.) were added into mixture, and the mixture was heated to 280° C. and kept for 10 h. The solid was cooled, ground, and washed by 100 mL of 10% HC and then water. The precipitate was purified by chromatograph to produce III-Zn-2: ¹H NMR (CDCl₃): 9.45 (d, 2H), 9.29 (dd, 2H), 8.14 (d, 4H), 8.01-8.06 (m, 4H), 7.91-7.95 (m, 4H), 7.85 (dd, 2H), 7.57 (dd, 2H), 7.35 (dd, 2H), 7.10 (dd, 2H), 6.84 (d, 2H),

3. Synthesis of I-Zn-2:

A mixture of 1,3-diaminoisoindoline (1.47 g), phenylacetic acid (1.4 g), and zinc oxide (0.4 g) was heated at 280° C. for 1 h. The resulting melt was cooled, ground, refluxed in 100 mL of 10% KOH for 30 min, filtered off, washed with water to neutral washing, and dried. The residue was purified by chromatography to produce I-Zn-2, and IV-Zn-2: I-Zn-2:1H NMR (CDCl3) 9.46 (d, 2H), 8.19 (d, 2H), 8.14 (d, 4H), 7.94-8.01 (m, 2H), 7.85-7.91 (m, 8H), 7.58 (t, 2H), 7.30-7.32 (m, 4H), 7.15-7.17 (m, 2H), 7.02-7.04 (m, 2H), 6.95 (d, 2H). IV-Zn-2: 1H NMR (DMSO) 9.56 (d, 2H), 9.46-9.49 (m, 4H), 8.27-8.30 (m, 4H), 8.20 (d, 2H), 8.16 (t, 1H), 8.06 (dd, 2H), 8.02 (dd, 2H), 7.70 (dd, 2H), 7.00 (d, 2H).

4. Synthesis of I-Pt-2:

A solution of 0.2 g of I-Zn-2 in 25 mL of sulfuric acid was allowed to stand at room temperature for 1 day and then poured into 100 ml of ice-water. The precipitate that formed was filtered off, washed with water to neutral washings. The residue was dried and purified by chromatography to form 1-H2-2. I-H12-2 and platinum(II) chloride (2 eq.) was resolved in DMSO. The mixture was heated to 120° C., and stand for 1 day, then poured into 100 mL of water. The precipitate that formed was filtered off, washed with water, and purified by chromatography to form I-Pt-2. ¹H NMR (500 MWz, CDCl₃) 9.55 (d, 2H), 8.24-8.26 (m, 6H), 7.97-8.02 (1.6H), 7.91 (dd, 4H), 7.88 (t, 2H), 7.29-7.33 (m, 4H), 7.20 (d, 2H), 7.15 (d, 2H), 7.05 (d, 2H).

5. Analysis of IV-Zn-2:

In another example, a device was assembled comprising an indium tin oxide (ITO) substrate, a layer of Zinc triazatetrabenzoporphyrin (IV-Zn-2), as described above, with a thickness between 5 nm and 20 nm, a 30 nm layer of C60, a 10 nm layer of (dihexyl-perylene tetracarboxylic diimide (PTCDI), a 14 nm layer of bathocuproine (BCP), and an aluminum layer. The current voltage characteristics of the device were then measured under 1 sun condition (AM1.5 @100 mW/cm²), the results of which are illustrated in FIG. 7. This data illustrates the suitability of the inventive azaporphyrin compounds as absorbers for organic photovoltaics applications.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1.-17. (canceled)

18. A compound represented by the formula: 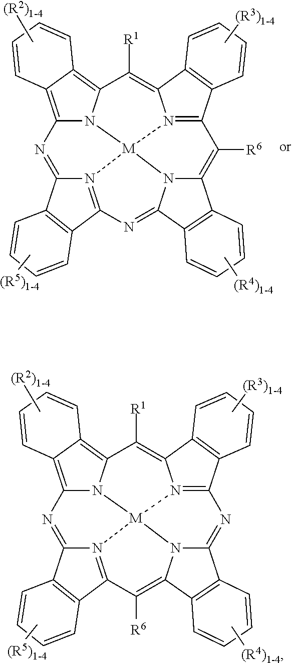
wherein M is Zn²⁺, Cu²⁺, Pd²⁺, Pt²⁺, Fe²⁺, Co²⁺, Ni²⁺, or Mg²⁺;
wherein R⁶ is optionally substituted aryl;
R¹ is hydrogen or optionally substituted aryl; and
wherein each of R², R³, R⁴, and R⁵ independently comprises four substituents independently selected from hydrogen, halide, and aryl,
wherein the compound is not

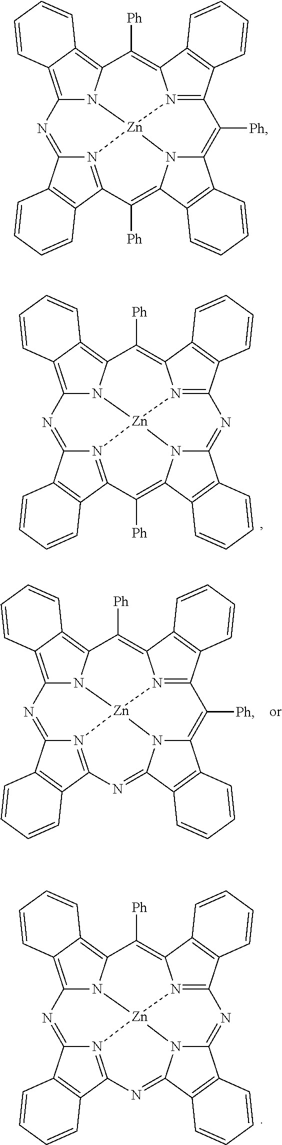

19. The compound of claim 18, wherein the compound is not

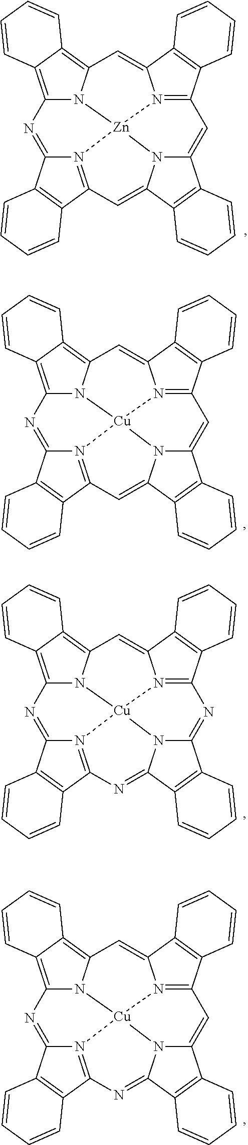

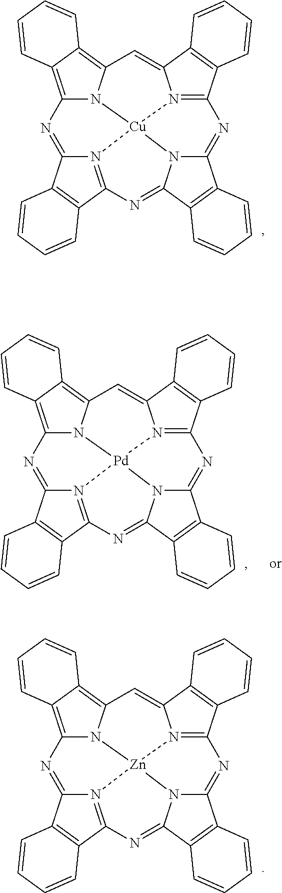

20. The compound of claim 18, wherein at least one of R², R³, R⁴, and R⁵ is not hydrogen.

21. The compound of claim 18, wherein M is Pd²⁺ or Pt²⁺.

22. The compound of claim 18, wherein each of R², R³, R⁴, and R⁵ independently comprises four hydrogens, provided that M is not Zn²⁺, Cu²⁺, or Pd²⁺.

23. The compound of claim 18, wherein M is Zn²⁺ or Cu²⁺.

24. The compound of claim 18, wherein R¹ is hydrogen.

25. The compound of claim 18, wherein R¹ is phenyl.

26. The compound of claim 18, wherein R¹ is bromophenyl.

27. The compound of claim 18, wherein one or more of R², R³, R⁴, and, R⁵ independently comprises four hydrogens.

28. The compound of claim 18, wherein R⁶, when present, is phenyl.

29. The compound of claim 18, wherein R⁶ is bromophenyl.

30. A device comprising the compound of claim 18.

31. The device of claim 30, wherein the device comprises at least one of a light emitting diode, organic thin solar cell, dye-sensitized solar cell, organic concentrator, solar hydrogen generation system, or a combination thereof.

32. A light emitting device comprising the compound of claim 18.

33. A photovoltaic device comprising the compound of claim 18.

34. A solar device comprising the compound of claim 18.
